(19) United States
(12) Patent Application Publication
(10) Pub. No.: US 2010/0233775 A1
(43) Pub. Date: Sep. 16, 2010

(54) System for the production of methane and other useful products and method of use

(75) Inventors: Bruce G. Schroder, Harrisburg, SD (US); Russell A. Pohl, Sioux Falls, SD (US)

Correspondence Address: Ekse Law Firm, PC, Water Fall Plaza, 6116 S. Lyncrest Avenue, Suite 105, Sioux Falls, SD, 57108, US

(73) Assignee: Tech V, LLC

(21) Appl. No.: 12/381,552

(22) Filed: Mar. 13, 2009

(51) Int. Cl. C12P 5/02; C12M 1/02; C12M 1/06; C12M 1/36

(52) U.S. Cl. 435/167; 435/289.1; 435/297.1; 435/303.3

(57) ABSTRACT

A system for the production of methane and other useful products and method of use for generating green natural gas as a fuel or component for use in the manufacturing of specialty chemicals. The system for the production of methane and other useful products and method of use includes a culture of methanogenic archea for converting an input material into an output material, a reactor vessel for housing at least a portion of the culture of methanogenic archea, an input material stream directed into the reactor vessel to facilitate contact between the input material stream and the methanogenic archea, and an output material stream created at least in part by the culture of methanogenic archea.

### REFERENCE TO RELATED APPLICATION

This application is related to application serial number TBD, entitled Methanogenic Reactor filed TBD.

### BACKGROUND OF THE INVENTION

1. Field of the Invention

The present invention relates to the generation of green natural gas through methanogenic conversion and more particularly pertains to a new system for the production of methane and other useful product and method of use for generating natural gas and cellular biomass from a variety of input material including biomass.

2. Description of the Prior Art

The use of methanogens and methanogenic processes is known in the prior art. More specifically, the systems utilizing methanogens to generate natural gas heretofore devised and utilized have generally been either capturing the gaseous output of naturally occurring systems, such as the Volta Experiment on Lake Como in 1778 or anaerobic digestion systems which consist basically of familiar, expected and obvious biological, chemical, and structural configurations, notwithstanding the myriad of designs encompassed by the crowded prior art which have been developed for the fulfillment of countless objectives and requirements.

The process of Methanogenesis is fairly well known. The following references provide a good working overview of the methanogenic process and are hereby incorporated by reference for all purposes: Archea: Molecular and Cellular Biology—Chapter 13 Methanogenesis, James G. Ferry and Kyle A. Kastead, Department of Biochemestry and Molecular Biology, The Pennsylvania State University, Universtiy Park, P A, edited by Ricardo Cavicchiolo, © 2007 ASM Press, Washington, DC; and Continuous Cultures Limited by a Gaseous Substrate: Development of a Simple, unstructured Mathematical Model and Experimental Verification with Methanobacterium thermoautotrophicum, N. Schill, W. M. van Gulik, D. Voisard, and U. von Stockar, Institute of Chemical Engineering, Swiss Federal Institute of Technology, Lausanne (EPFL), CH-1015 Lausanne, Switzerland, Biotechnology and Bioengineering, Vol. 51, P6450658 (1996) John Wiley & Sons, Inc.

Illustrative examples of the types of systems known in the prior art include anaerobic digestion systems and U.S. Pat. Nos. 1,940,944; 2,097,454; 3,640,846; 4,722,741; 5,821,111 and application no. PCT/US07/71138.

In these respects, the system for the production of methane and other useful product and method of use according to the present invention substantially departs from the conventional concepts and designs of the prior art, and in so doing provides an apparatus primarily developed for the purpose of generating green natural gas and cellular biomass from a variety of source materials including biomass.

### SUMMARY OF THE INVENTION

To promote the development of renewable energy sources, the United States government has identified a “billion ton” goal of biomass production per year. At present the largest single component of that supply is corn stover. It is widely accepted that adverse ecological effects of corn production such as the anoxic zone in the Caribbean will encourage biomass production from other sources. Chief among these alternatives will be perennial grasses such as switchgrass and native prairie grasses. Testing currently underway on novel, high yield grasses such as miscanthus points to the prospect of biomass production in lieu of conventional crops on marginal lands.

Despite these developments on the production side there remain critical issues on the conversion of these biomass sources to useable forms as substitutes for fossil fuels.

Pelletizing improves the handling characteristics of biomass, but adds enough cost to the resulting fuel cost to largely eliminate any fuel cost advantage. In addition, biomass fuels burn dirty, producing sulfur and nitrogen oxides and hydrogen chloride. Equipment to burn these fuels is expensive and air permitting remains problematic. A clean solution to these limitations would be to convert biomass into pipeline quality biomethane near the point of origin for transmission to existing natural gas customers via existing natural gas pipelines. This same process can also supply biomethane to specialty chemical facilities for the production of green specialty chemicals, including but not limited to “green plastics”. Further, the present invention also generates cellular biomass, which may be utilized as a food or nutrient for livestock and humans

The two primary routes to biomethane currently recognized are anaerobic digestion and thermochemical conversion. A third process for the conversion of biomass to liquid fuels is being pursued which involves enzymatic breakdown of cellulose and hemicelluloses into fermentable sugars. While these processes are effective on some feedstocks and at some capacities, none of them provide a fully satisfactory route to biomass use.

To understand why this is so, it is helpful to understand the progression of plant composition during the growing season. The three primary structures in a plant are cellulose, hemicelluloses, and lignin. These compounds are in turn polymers of 6-carbon sugars, 5-carbon sugars, and phenolics respectively. As the plant matures, there is a progressive conversion of cellulose and hemicelluloses to lignin. This is reflected in the decrease of total digestable nutrients and the increase of acid detergent fiber content.

Anaerobic digestion uses mixed cultures of microbes to break down biomass into fermentable sugars, amino acids, and organic acids. This process is multi-step and is subject to upset by over-production of organic acids which kill the methanogenic organisims. The great benefit of anaerobic digestion is that it is generally recognized as specific, producing methane and carbon dioxide in a readily recoverable form.

Anaerobic digestion of grasses and corn stover has been extensively studied. Mahert (Mahert, Pia, et al, “Batch and Semi-continuous Biogas Production from Different Grass Species”. December 2005) and others have studied the potential for biomethane production from various grasses. The chief finding of this work is that while biomethane can be produced by this route, the required digester volume per unit of energy produced is uneconomical. In addition, the grasses must be harvested at or before full bloom. Corn stover is substantially limited and in some instances near impervious to anaerobic digestion.

A further disadvantage of anaerobic digestion of grasses is that when native or prairie grass is cut before October, the yield the following year is half or less of what is expected. This appears to be related to the manner in which nutrients are returned to the root structure after frost.

Thermochemcial conversion of biomass to biomethane and liquid fuels is a proven technology base on some old coal chemistry. A large scale coal to natural gas plant at Beulah, N. Dak. has been in operation since the late 1980's. The chief limitation of this technology is that it strongly favors large scale operations, generally over 400 tons per day.

Enzymatic processes to break down biomass to fermentable sugars remain an elusive and expensive undertaking. Even if successful, however, enzymatic processes are likely to be highly specific to certain species and perhaps even varieties within species due to their high specificity. One of the objectives associated with biomass production is promotion of multiple species cultivation. Highly specific enzyme processes will tend to promote monocultures and leave the ecosystem no richer than a corn/soybean mix.

As the foregoing shows, there is room for development of a novel process which will address the limitations of all the current options. Such a process will have at least some of the following characteristics:
1) It will produce a fuel which is directly compatible with existing energy distribution and use equipment;
2) It will use a variety of feed stocks ranging from corn stover to perennial grasses to wood without loss of yield per ton of saleable energy;
3) It will utilize feedstock harvested late in season and preferable after frost;
4) It will be economical at a scale of 200 ton per day or less;
5) It will be modular to allow initial construction and expansion as the biomass supply chain becomes established and more efficient and
6) It will produce cellular biomass that can have useful and economic value.

The present invention provides each of these advantages by using a hybrid process which combines the flexibility and power of gasification with the specificity of anaerobic digestion, and with improved efficiency and higher production rates than anaerobic digestion. The gasification step overcomes biomass species and variety variations producing uniform, readily fermentable feedstock to the reactor. The culture in the reactor is efficient and specific producing only methane, cellular biomass, and water as its co-products.

The present invention utilizes a wide variety of feedstocks ranging from crop residues, low value co-products from agriculture processing and energy crops such as switchgrass and corn stover, waste wood products, and other similar biomass sources. The raw materials may be processed such as being reduced to a uniform size and moisture content (preferably very low) prior to gasification. The gassification process converts the biomass into an intermediate gas stream known as syngas or synthesis gas. The syngas, after going through a heat recovery process, may be directed through a filtering system and or a water gas shift prior to being directed into the reactor vessel for conversion by the methanogenic culture into methane.

It is important to note that while the present invention is directed towards providing green natural gas from biomass, the same process can be done with municipal or landfill wastes or nonconventional carbon and hydrogen sources (collectively “landfill waste”). The use of the present invention with landfill wastes as the feed stock would allow the reclamation of hundreds of thousands of acres currently used as landfills. If landfill wastes are utilized as a feedstock, the filtering and cleanup process after gasification can be much more complex than that required for biomass feedstock.

### BRIEF DESCRIPTION OF THE DRAWINGS

The invention will be better understood and objects of the invention will become apparent when consideration is given to the following detailed description thereof. Such description makes reference to the annexed drawings wherein:

FIG. 1 is a schematic functional block diagram of a new System For The Production Of Methane And Other Useful Products And Method Of Use according to the present invention.

FIG. 2 is a schematic functional block diagram of an embodiment of the present invention.

FIG. 3 is a schematic functional block diagram of the agitation system of the present invention.

FIG. 4 is a schematic functional block diagram of the recirculation system of the present invention.

FIG. 5 is a schematic functional block diagram of the pH control system of the present invention.

FIG. 6 is a schematic flow diagram of the present invention.

### DESCRIPTION OF PREFERRED EMBODIMENTS

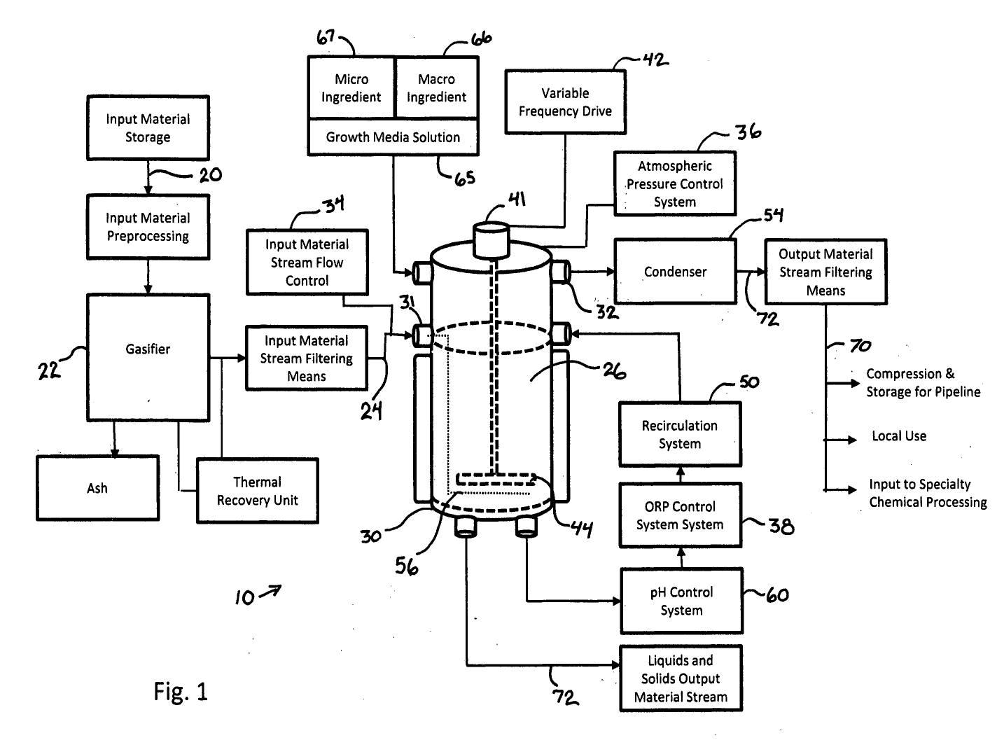

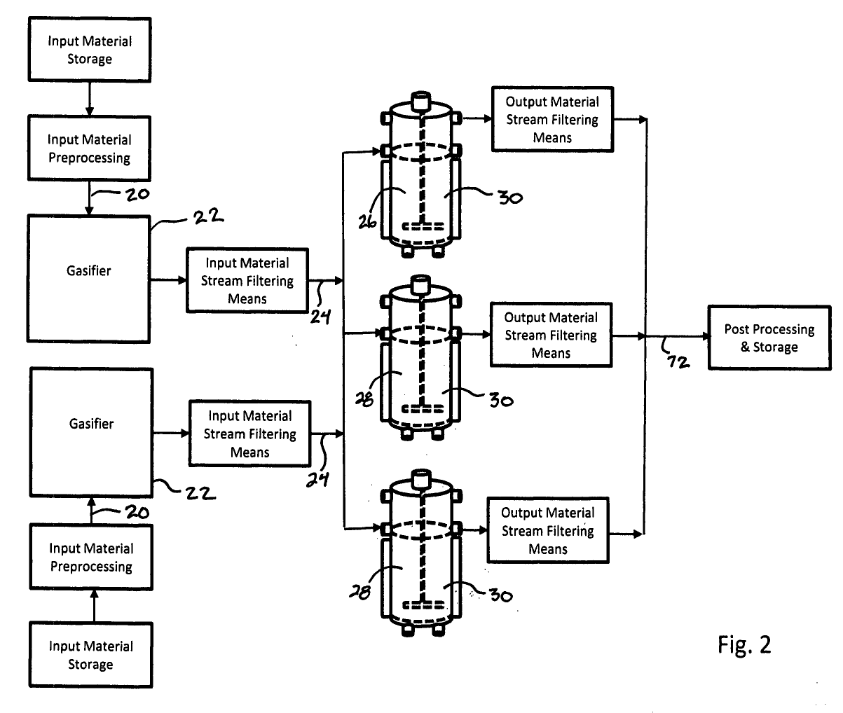

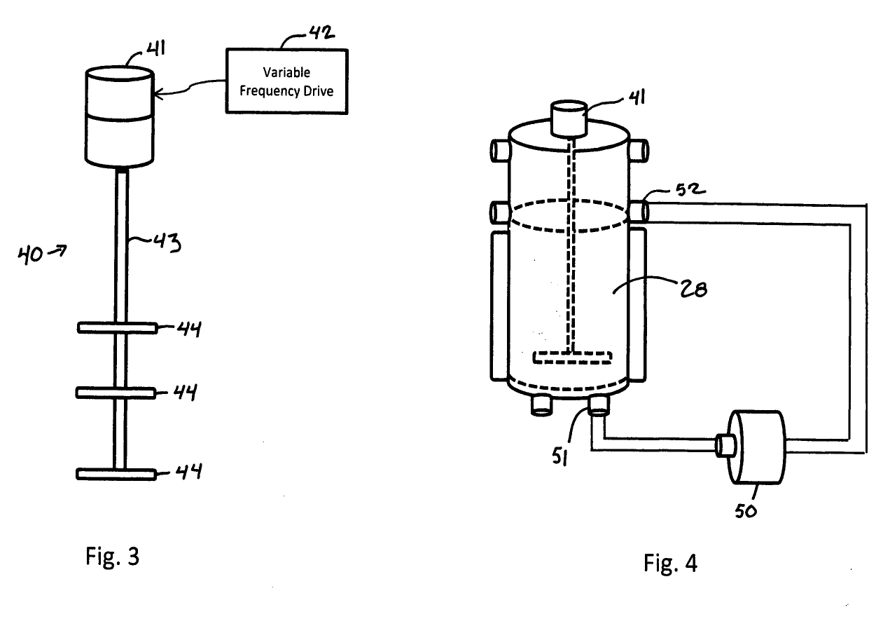

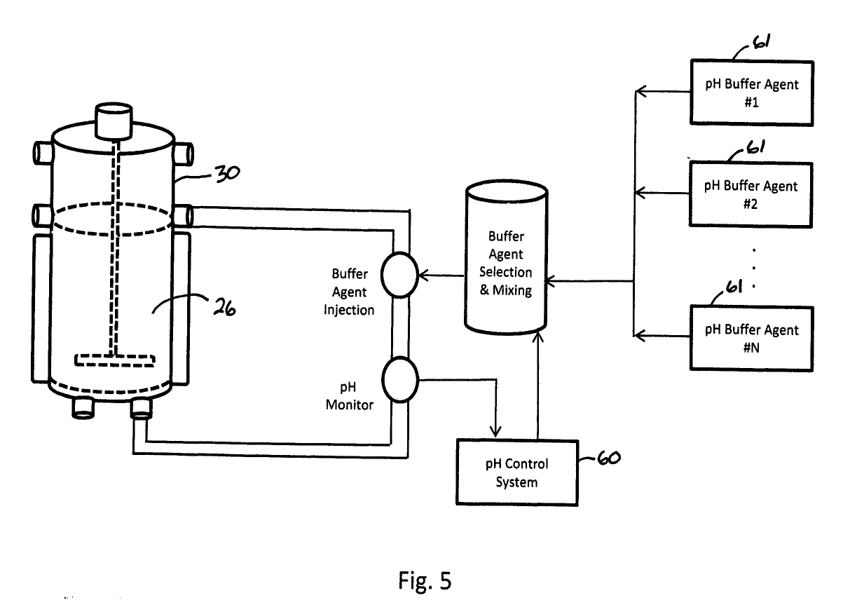

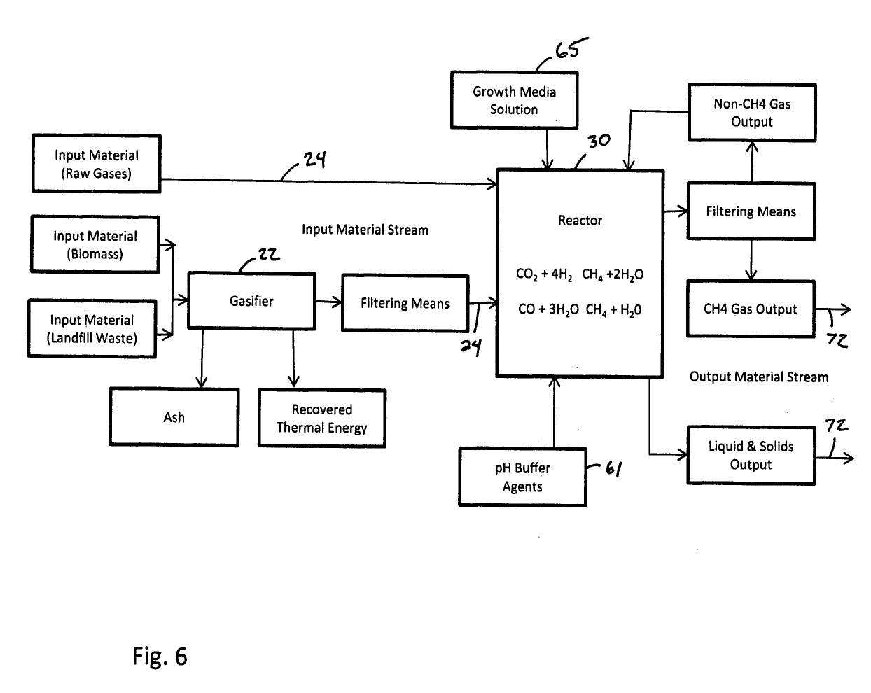

With reference now to the drawings, and in particular to FIGS. 1 through 6 thereof, a new System for the production of methane and other useful products and method of use embodying the principles and concepts of the present invention and generally designated by the reference numeral 10 will be described.

As best illustrated in FIGS. 1 through 6, the System for the production of methane and other useful products and method of use 10 generally comprises a culture of methanogenic archea 26 for converting an input material 20into an output material 70, a reactor vessel 30 for housing at least a portion of the culture of methanogenic archea 26, an input material stream 24 directed into the reactor vessel 30 to facilitate contact between the input material stream 24 and the methanogenic archea 26, and an output material stream 72 created at least in part by the culture of methanogenic archea 26.

In at least one embodiment, the present invention includes at least one culture of methanogenic archea 26 (“methanogens”), at least one reactor vessel 30, at least one input material stream 24, and at least one output material stream 72.

The methanogens convert an input material 20into an output material, including methane and cellular biomass. Typically, the present invention uses primarily a gas mixture for the input stream and generates at least a gas and excess biological material as the output materials.

At least one reactor vessel 30 is used for housing at least a portion of the culture of methanogenic archea 26. The present invention also has at least one embodiment in which multiple reactor vessels 30 are used in parallel. This type of parallel arrangement has the benefits of scaling the total reactor volume as desired by adding additional reactor vessels 30, providing the ability to continue operations during maintenance procedures, and advantageous vessel sizing.

The input material stream 24 is directed into the reactor vessel 30 to facilitate contact between the input material stream 24 and the methanogenic archea 26. While conceptualized as an input material stream 24 the present invention also has at least one embodiment wherein multiple input streams are utilized. As an illustrative example only, one such embodiment may occur when sequestered CO₂ is used along with other input materials. In this example, the sequestered CO₂ could be directed into the reactor vessel 30 separate from the other input materials. Further, the present invention also includes at least one embodiment wherein H₂, CO, CO₂, and/or H₂S are directed into the reactor vessel 30 as separate input streams.

The output material stream 72 is created at least in part by bringing the methanogens into contact with the input material stream 24. Preferably, this contact occurs at a molecular scale. The output material stream 72 may also include multiple output material streams 72 such as a gas stream, a solids stream, and a liquids stream. The liquids stream may include particulate matter and dissolved gases.

In an embodiment each one of the reactor vessels 30 includes at least one input material stream port 31 for operationally coupling the reactor vessel 30 to a source of the input material stream 24, at least one output material stream port 32 for facilitating removal of the output material stream 72.

Preferably each one of the reactor vessel 30s also may include an agitation system 40, a recirculation system, a pH adjustment system 60, a condenser 54, an input material stream flow control 34, atmoshpheric pressure control system 36, and an Oxidation Reduction Potential control system 38. The agitation system 40 is at least partially positioned within the reactor vessel 30 and is used for enhancing contact between the input material stream 24 and the culture of methanogenic archea 26 and for reducing foaming within the reactor vessel 30. In some cases an anti-foaming agent may be added into the reactor to further reduce foaming. The recirculation system 50 is also used to enhance contact between the input material stream 24 and the culture of methanogenic archea 26. The pH adjustment system 60 facilitates the maintenance of a pH of the methanogenic archea 26 combined with a mixture of the input material stream 24 and the output material stream 72. Typically if the pH falls below a predetermined level, a buffer solution is added into the reactor vessel 30, and if the pH increases above a second predetermined level, additional CO2 is introduced into the reactor vessel 30. The condenser 54 is preferably environmentally coupled to the output material stream 72 port 32, and allows a gaseous portion of the output material stream 72 to be separated from a non-gaseous portion of the output material stream 72. In at least one embodiment the condenser 54 is sixed such that the volume of the condenser 54 to the volume of the reactor is between 1:20 and 1:160. The input material stream flow control 34 is used to control the type and rate of material in the input material stream 24 being directed into the reactor vessel 30. The atmospheric pressure adjustment system facilitates the control and maintenance of atmospheric pressure within the reactor vessel 30. Typically the pressure utilized within the reactor vessel 30 is between 0.5 and 7 atmospheres. In at least one embodiment at “normal” atmospheric pressure the reactor in operation may maintain between 2 and 7 PSI of back pressure. The oxidation reduction potential adjustment system facilitates the maintenance of an oxidation reduction potential (“ORP”) of the methanogenic archea 26 combined with a mixture of the input material stream 24 and the output material stream 72. Typically if the ORP falls outside of a desired range a predetermined quantity of H₂ or H₂S is introduced into the reactor vessel 30.

In at least one embodiment, at least a portion of the input material stream 24 is the output of a gasifier 22. The gasifier 22 type may include steam reforming, air swept, or oxygen swept dependent at least in part on the type of materials to be gasified. The present invention accommodates a wide range of gasifier 22 input materials including corn stover, switch grass, wood waste products, municipal or landfill wastes, and other similar materials.

The input material stream 24 or streams directed into the reactor vessel 30 may include any of the following combinations:
1) Carbon Dioxide and Hydrogen;
2) Carbon Monoxide and Hydrogen;
3) Carbon Dioxide and Carbon Monoxide;
4) Carbon Dioxide, Carbon Monoxide, and Hydrogen;
5) Carbon Dioxide, Hydrogen, and Hydrogen Sulfide;
6) Carbon Dioxide, Hydrogen, Hydrogen Sulfide and Nitrogen;
7) Carbon Dioxide, Hydrogen, Hydrogen Sulfide, Nitrogen, and Oxygen;
8) Carbon Dioxide, Hydrogen, Hydrogen Sulfide, Nitrogen, and Oxygen;
9) Carbon Dioxide, Hydrogen, Hydrogen Sulfide, Nitrogen, Carbon Monoxide, and Oxygen;
10) Carbon Dioxide, Hydrogen, Nitrogen, Carbon Monoxide, and Oxygen;
11) Carbon Dioxide, Hydrogen, Carbon Monoxide, and Oxygen;
12) Carbon Dioxide, Hydrogen, Carbon Monoxide, and Nitrogen;
13) Carbon Dioxide, Hydrogen, Carbon Monoxide, and Hydrogen Sulfide;
14) Carbon Dioxide, Hydrogen, Carbon Monoxide, Hydrogen Sulfide, and Oxygen;
15) Carbon Monoxide, Hydrogen, and Hydrogen Sulfide;
16) Carbon Monoxide, Hydrogen, and Nitrogen;
17) Carbon Monoxide, Hydrogen, Hydrogen Sulfide, and Nitrogen;
18) Carbon Monoxide, Hydrogen, Hydrogen Sulfide, Nitrogen, and Oxygen;
19) Carbon Monoxide, Hydrogen, Nitrogen, and Oxygen;
20) Carbon Monoxide, Hydrogen, Hydrogen Sulfide, and Oxygen;
21) Carbon Monoxide, Hydrogen, Nitrogen, and Oxygen;

As may be readily appreciated from reviewing the above listing, the present invention has significant tolerance to variations in the input material stream 24, and as such gas cleanup prior to introduction into the reactor vessel 30 may be significantly reduced or potentially eliminated. Further, for each of the combinations listed above, the input material stream 24 may be directed into the reactor vessel 30 as a single mixed stream, or as a combination of multiple streams each directed into the reactor vessel 30.

For sustained operation, the present invention may also include a growth media solution 65 for promoting the ongoing growth of the culture of methanogenic archea 26, as well as enhancing the production of methane by the methanogenic archea 26.

In an embodiment the growth media solution 65 includes both a macro ingredient solution 66 and a micro ingredient solution 67. Preferably, the macro ingredient solution 66 comprises KH₂PO₄, NH₄CL, and NaCl. More preferably, the macro ingredient solution 66 comprises 75 to 300 grams of KH₂PO₄, 350 to 1600 grams of NH₄CL, and 30 to 130 grams of NaCl dissolved in 20 to 40 gallons of deionized water. In at least one embodiment, the macro ingredient solution 66 comprises approximately 153.8 grams of KH₂PO₄, approximately 725.3 grams of NH₄CL, and approximately 66.0 grams of NaCl dissolved in approximately 30 gallons of water.

In a preferred embodiment the micro ingredient solution 67 comprises Na2 nitrilotriacetates, MgCl₂-6H₂O, FeSO₄-7H₂O, CoCl₂-6H₂o, Na₂MoO₄-2H₂O, NiCl₂-6H₂o, Na₂SeO₃, Na₂WO₄-2H₂O. More preferably the micro ingredient solution 67 comprises 35 to 150 grams per liter of Na₂ nitrilotriacetates, 25 to 100 grams per liter of MgCl₂-6H₂O, 6 to 30 grams per liter of FeSO₄-7H₂O, 0.07 to 0.30 grams per liter of CoCl₂-6H₂O, 0.07 to 0.30 grams per liter of Na₂MoO₄-2H₂O, 0.15 to 0.60 grams per liter of NiCl₂-6H₂O, 0.01 to 0.1 grams per liter of Na₂SeO₃, 0.40 to 1.7 grams per liter Na₂WO₄-2H₂O. In at least one embodiment, the micro ingredient solution 67 comprises approximately 70.5 grams per liter of Na₂ nitrilotriacetates, approximately 50.8 grams per liter of MgCl₂-6H₂O, approximately 13.9 grams per liter of FeSO₄-7H₂O, approximately 0.15 grams per liter of CoCl₂-6H₂O, approximately 0.15 grams per liter of Na₂MoO₄-2H₂O, approximately 0.30 grams per liter of NiCl₂-6H₂O, approximately 0.04 grams per liter of Na₂SeO₃, approximately 0.82 grams per liter Na₂WO₄-2H₂O.

In an embodiment the micro ingredient solution 67 is prepared using deaerated water and maintained in anoxic condition in order to maintain iron ions as iron+3.

In at least one embodiment, the growth media solution 65 is prepared by first preparing the macro ingredient solution 66 under normal atmospheric conditions and then deaerating the macro ingredient solution 66. Concurrently, the micro ingredient solution 67 is prepared under anoxic condition. The micro ingredient solution 67 is added to the deaerated macro ingredient solution 66.

In a further embodiment the micro ingredient solution 67 is added to the deaerated macro ingredient solution 66 at a ratio between 1 part micro ingredient solution 67 to 100 to 400 parts macro ingredient solution 66.

In still a further embodiment the micro ingredient solution 67 is added to the deaerated macro ingredient solution 66 at a ratio of 1 part micro ingredient solution 67 to 250 parts macro ingredient solution 66.

In an embodiment the media growth solution is directed into the reactor vessel 30 through at least one media input port.

Preferably, the media growth solution is maintained under a nitrogen blanket.

In an embodiment the input material stream 24 comprises at least in part carbon dioxide and a percentage of carbon dioxide is converted into cellular biomass during exposure to the culture of methanogenic archea 26 is less than 20 percent.

In an embodiment the input material stream 24 comprises at least in part carbon dioxide and a percentage of carbon dioxide is converted into biomass during exposure to the culture of methanogenic archea 26 is between approximately 5 and 15 percent inclusive.

In an embodiment excess or dead biomass is selectively removed from the reactor through at least one biomass elimination port positioned on a lower portion of the reactor vessel 30.

In an embodiment the pH adjustment system 60 further comprises a pH buffer agent 61. The buffer agent 61 may be sodium hydroxide, potassium hydroxide, calcium hydroxide, sodium bicarbonate, ammonia, ammonium, or ammonia nitrate. Preferably the buffer solution is prepared using deaerated water and maintained under nitrogen until introduction into the reactor vessel 30.

In at least one embodiment the buffer agent 61 is prepared as approximately a 1.0 Normal solution.

In another embodiment the pH buffer agent 61 is prepared as less than a 1.0 Normal solution.

In still a further embodiment, multiple buffer solutions, selected from the list of buffer solutions provided above are available, and a specific buffer solution or combination of buffer solutions are used based at least in part upon the rate of change of the pH within the reactor vessel 30. If the pH falls out of a predetermined desirable range, typically 7.6 to 8.6, then buffer solution is added to the reactor vessel 30.

In an embodiment the input material stream 24 is routed into the reactor vessel 30 at a rate of 0.5 to 4.0 scfm per 5 cubic feet of reactor vessel 30 volume.

In an embodiment the input material stream 24 is routed into the reactor vessel 30 at a rate of 1.0 to 2.6 scfm per 5 cubic feet of reactor vessel 30 volume.

In an embodiment the input material stream 24 is routed into the reactor vessel 30 at a rate of approximately 1.9 to 2.6 scfm per 5 cubic feet of reactor vessel 30 volume.

In an embodiment the input material stream 24 is routed into the reactor vessel 30 and through a sparger 56 positioned within the reactor vessel 30.

In an embodiment the sparger 56 creates bubbles approximately 1 micron to 10 microns in diameter.

In an embodiment the output material stream 72 is generated at a rate of between 10 and 150 volumes per effective reactor volume per day (“VVD”). As an illustrative example only, let us assume that a 1000 cubic foot reactor is used. Further let us assume that there is a headspace within the reactor that occupies approximately 200 cubic feet. Thus the effective reactor volume is 800 cubic feet. If the output material stream 72 for this illustrative example is produced at 100 VVD, then the resulting output would be 80,000 cubic feet, per day.

In another embodiment the output material stream 72 is generated at a rate of between 35 and 100 VVD.

In still another embodiment the output material stream 72 is generated at a rate of between 45 and 70 VVD.

In at least one embodiment, the input material stream 24, whether directed into the reactor as a blended gas or as individual gas streams, may include approximately four parts hydrogen to one part carbon dioxide. In the methanogenic reaction which follows, approximately 5 to 15% of the carbon dioxide is converted to cellular biomass through contact with the culture of methanogenic archea and the output material stream 72 comprises approximately 60 to 85% CH4. The output material stream 72 may also include hydrogen.

In another embodiment, the input material stream 24, whether directed into the reactor as a blended gas stream or as individual gas streams, may include approximately two parts hydrogen to one part carbon dioxide. In the methanogenic reactor which follows, approximately 5 to 15% of the carbon dioxide is converted to cellular biomass through contact with the culture of methanogenic archea 26, and the output material stream 72 comprises approximately 50 to 85% CH4. The output material stream 72 may also include carbon dioxide.

In yet another embodiment, the input material stream 24 whether directed into the reactor as a blended gas or as individual gas streams, or as multiple streams at least one of which comprises a combination of gases, may include between approximately two parts hydrogen, approximately five parts hydrogen and approximately one part carbon dioxide. In the methanogenic reaction which follows, approximately 5 to 15% of the carbon dioxide is converted to cellular biomass through contact with the culture of methanogenic archea 26. And the output material stream 72 comprises approximately 50 to 85% CH4. The output material stream 72 may also include carbon dioxide and/or hydrogen.

Depending upon the composition of the input material stream 24 provided, it may be desirous to direct the input material stream 24 through a gas filtering means 58 prior to directing the input material stream 24 into the reactor vessel 30. Several types of gas filtering means 58 are known, and may be selected at least in part based upon the composition of the input material stream 24. Examples of such gas filtering means 58 include, but are not limited to: Water Gas Shift Reactors, Pressure Swing Adsorption Reactors, Vacuum Swing Adsorption Reactor, and Membrane Filters.

Similarly, the output material stream 72 may be directed through a gas filtering means 58, prior to being stored, compressed, or otherwise utilized. The gas filtering means 58 for the output material stream 72 may be any one of a number of gas filtering means 58, including but not limited to: Water Gas Shift Reactors, Pressure Swing Adsorption Reactors, Vacuum Swing Adsorption Reactors, and Membrane Filters.

In at least one embodiment the output stream filtering means includes a methane output and a recycling output. The recycling output may be directed back into the reactor vessel 30.

The system may also include a thermal conditioning assembly operationally coupled to the reactor vessel 30, for helping to maintain an internal temperature for the reactor vessel 30 between 55 and 70 degrees Celsius, and more preferably, between 60 and 65 degrees Celsius.

The system may also include a second culture of methanogenic archea 28 for converting an input material 20into an output material. This second culture may be inoculated into the reactor vessel 30, or may be generated within the reactor vessel 30 during a prolonged operational phase for the system.

The system may also make use of multiple reactor vessels 30, with the reactor vessels 30 being connected in parallel between the input material stream 24 and output material stream 72. Each one of the multiple reactor vessels 30 may enclose an associated culture of methanogenic archea 26. The cultures of the multiple reactors need not necessarily be the same strain or type of methanogenic archea 26.

In at least one embodiment the input material stream 24 is directed into the reactor vessel 30 and the output material stream 72 is released from the reactor vessel 30 in a continuous manner.

In another embodiment the input material stream 24 is directed into the reactor vessel 30 periodically.

In still another embodiment the output material stream 72 is released from the reactor vessel 30 periodically.

The agitation system 40 may include an agitation drive means 41, and an impeller 44 operationally coupled to the agitation drive means 41. As an illustrative example of an agitation drive means 41 as contemplated by the present invention a motor electrically coupled to a variable frequency drive to control the speed of the motor may be magnetically coupled to an agitation shaft 43 positioned within the reactor. The impeller 44 is thus operationally coupled to the motor.

In an embodiment the impeller 44 rotates at between 1100 and 2100 rpm during normal operation of the reactor. More preferable the impeller 44 rotates at between 1500 and 1800 rpm during normal operation.

In another embodiment the impeller 44 rotates at greater than 110% of the resonance of the reactor vessel 30.

It is important to note that the tip speed of the impeller 44 is critical and thus proper sizing is important. In at least one embodiment the tip speed of the impeller 44 is between 5 and 45 mph.

The recirculation system 50 selectively removes a portion of a combination of the culture of methanogenic archea 26 and the growth media through at least one recirculation outlet port 51 of the reactor vessel 30. The recirculation system 50 returns the portion of the combination into the reactor through at least one recirculation inlet port 52 of the reactor vessel 30.

In an embodiment the selective removal and returning of the portion of the combination is done at a rate of between 5 and 50 percent of the reactor volume per hour.

In another embodiment the selective removal and returning of the portion of the combination is done at a rate of between 10 and 20 percent of the reactor volume per hour.

In an embodiment the culture of methanogenic archea 26 comprises methanobacterim thermoautotrophicum or methanothermobacter thermautotrophicus.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A system for using methanogenic archea for the creation of useful products comprising:
a culture of methanogenic archea for converting an input material into an output material;
a reactor vessel for housing at least a portion of the culture of methanogenic archea;
an input material stream, said input material stream being directed into said reactor vessel to facilitate contact between said input material stream and said methanogenic archea; and
an output material stream created at least in part by the culture of methanogenic archea.

2. The system of claim 1, wherein said reactor vessel further comprises:
an input material stream port for operationally coupling said reactor vessel to a source of said input material stream; and
an output material stream port for facilitating removal of said output material stream.

3. The system of claim 1, wherein said reactor vessel further comprises an agitation system, said agitation system being at least partially positioned within said reactor vessel, said agitation system enhancing contact between said input material stream and said culture of methanogenic archea.

4. The system of claim 1, wherein said reactor vessel further comprises a recirculation system, said recirculation system enhancing contact between said input material stream and said culture of methanogenic archea.

5. The system of claim 1, wherein said reactor vessel further comprises a pH adjustment system, said pH adjustment system facilitating the maintenance of a pH of the methanogenic archea combined with a mixture of said input material stream and said output material stream.

6. The system of claim 2, further comprising a condenser environmentally coupled to said output material stream port, said condenser allowing a gaseous portion of said output material stream to be separated from a non-gaseous portion of said output material stream.

7. The system of claim 1, further comprising an input material stream flow control whereby the flow of the input material stream into said reactor vessel may be controlled.

8. The System of claim 1, further comprising an atmospheric pressure adjustment system, said atmospheric pressure adjustment system facilitating the control and maintenance of atmospheric pressure within said reactor vessel.

9. The system of claim 1, further comprising an oxidation reduction potential adjustment system, said oxidation reduction potential adjustment system facilitating the maintenance of an oxidation reduction potential of the methanogenic archea combined with a mixture of said input material stream and said output material stream.

10. The system of claim 1, further comprising:
said input material stream comprises approximately 2 parts hydrogen to one part carbon dioxide;
said input material stream is routed into said reactor vessel at a rate of 0.5 to 4.0 scfm per 5 cubic feet of reactor vessel volume;
wherein approximately 5 to 15% of the carbon dioxide is converted to biomass through contact with the culture of methanogenic archea;
said output material stream is generated at a rate of between 10 and 150 VVD; and
wherein said output material stream comprises approximately 50 to 85% CH4.

11. A system for using methanogenic archea for the creation of useful products comprising:
a culture of methanogenic archea for converting an input material into an output material;
at least one reactor vessel for housing at least a portion of the culture of methanogenic archea;
at least one input material stream, said input material stream being directed into said reactor vessel to facilitate contact between said input material stream and said methanogenic archea;
at least one output material stream created at least in part by the culture of methanogenic archea;
wherein each one of said at least one reactor vessel further comprises:
at least one input material stream port for operationally coupling said reactor vessel to a source of said input material stream;
at least one output material stream port for facilitating removal of said output material stream;
an agitation system, said agitation system being at least partially positioned within said reactor vessel, said agitation system enhancing contact between said input material stream and said culture of methanogenic archea;
a recirculation system, said recirculation system enhancing contact between said input material stream and said culture of methanogenic archea;
a pH adjustment system, said pH adjustment system facilitating the maintenance of a pH of the methanogenic archea combined with a mixture of said input material stream and said output material stream;
a condenser environmentally coupled to said output material stream port, said condenser allowing a gaseous portion of said output material stream to be separated from a non-gaseous portion of said output material stream; and
an input material stream flow control whereby the flow of the input material stream into said reactor vessel may be controlled.

12. The system of claim 11, wherein said input material stream comprises the output of a gasifier.

13. The system of claim 11, wherein said input material stream comprises Carbon Dioxide and Hydrogen.

14. The system of claim 11, wherein said input material stream comprises Carbon Monoxide and Hydrogen.

15. The system of claim 11, wherein said input material stream comprises Carbon Dioxide and Carbon Monoxide.

16. The system of claim 11, wherein said input material stream comprises Carbon Dioxide, Carbon Monoxide, and Hydrogen.

17. The system of claim 11, wherein said input material stream comprises Carbon Dioxide, Hydrogen, and Hydrogen Sulfide.

18. The system of claim 11, wherein said input material stream comprises Carbon Dioxide, Hydrogen, Hydrogen Sulfide and Nitrogen.

19. The system of claim 11, wherein said input material stream comprises Carbon Dioxide, Hydrogen, Hydrogen Sulfide, Nitrogen, and Oxygen.

20. The system of claim 11, wherein said input material stream comprises Carbon Dioxide, Hydrogen, Hydrogen Sulfide, Nitrogen, and Oxygen.

21. The system of claim 11, wherein said input material stream comprises Carbon Dioxide, Hydrogen, Hydrogen Sulfide, Nitrogen, Carbon Monoxide, and Oxygen.

22. The system of claim 11, wherein said input material stream comprises Carbon Dioxide, hydrogen, Nitrogen, Carbon Monoxide, and Oxygen.

23. The system of claim 11, wherein said input material stream comprises Carbon Dioxide, Hydrogen, Carbon Monoxide, and Oxygen.

24. The system of claim 11, wherein said input material stream comprises Carbon Dioxide, Hydrogen, Carbon Monoxide, and Nitrogen.

25. The system of claim 11, wherein said input material stream comprises Carbon Dioxide, Hydrogen, Carbon Monoxide, and Hydrogen Sulfide.

26. The system of claim 11, wherein said input material stream comprises Carbon Dioxide, Hydrogen, Carbon Monoxide, Hydrogen Sulfide, and Oxygen.

27. The system of claim 11, wherein said input material stream comprises Carbon Monoxide, Hydrogen, and Hydrogen Sulfide.

28. The system of claim 11, wherein said input material stream comprises Carbon Monoxide, Hydrogen, and Nitrogen.

29. The system of claim 11, wherein said input material stream comprises Carbon Monoxide, Hydrogen, Hydrogen Sulfide, and Nitrogen.

30. The system of claim 11, wherein said input material stream comprises Carbon Monoxide, Hydrogen, Hydrogen Sulfide, Nitrogen, and Oxygen.

31. The system of claim 11, wherein said input material stream comprises Carbon Monoxide, Hydrogen, Nitrogen, and Oxygen.

32. The system of claim 11, wherein said input material stream comprises Carbon Monoxide, Hydrogen, Hydrogen Sulfide, and Oxygen.

33. The system of claim 11, wherein said input material stream comprises Carbon Monoxide, Hydrogen, Nitrogen, and Oxygen.

34. The system of claim 1, further comprising a growth media solution, said growth media solution being for promoting the ongoing growth of said culture of methanogenic archea.

35. The system of claim 11, further comprising a growth media solution, said growth media solution being for promoting the ongoing growth of said culture of methanogenic archea.

36. The system of claim 35, wherein said growth media solution further comprises a macro ingredient solution and a micro ingredient solution.

37. The system of claim 36, wherein said macro ingredient solution comprises KH2PO4, NH4CL, and NaCl.

38. The system of claim 36, wherein said macro ingredient solution comprises 75 to 300 grams of KH2PO4, 350 to 1600 grams of NH4CL, and 30 to 130 grams of NaCl dissolved in 20 to 40 gallons of deionized water.

39. The system of claim 36, wherein said macro ingredient solution comprises approximately 153.8 grams of KH₂PO₄, approximately 725.3 grams of NH₄CL, and approximately 66.0 grams of NaCl dissolved in approximately 30 gallons of water.

40. The system of claim 36, wherein said micro ingredient solution comprises Na2 nitrilotriacetates, MgCl₂-6H₂O, FeSO₄-7H₂O, CoCl₂-6H₂O, Na₂MoO₄-2H₂O, NiCl₂-6H₂O, Na₂SeO₃, Na₂WO₄-2H₂O.

41. The system of claim 36, wherein said micro ingredient solution comprises Approximately 70.5 grams per liter of Na₂ nitrilotriacetates, approximately 50.8 grams per liter of MgCl₂-6H2o, Approximately 13.9 grams per liter of FeSO₄-7H₂O, approximately 0.15 grams per liter of CoCl₂-6H₂O, approximately 0.15 grams per liter of Na₂MoO₄-2H₂O, approximately 0.30 grams per liter of NiCl₂-6H₂O, approximately 0.04 grams per liter of Na₂SeO₃, approximately 0.82 grams per liter Na₂WO₄-2H₂O.

42. The system of claim 36, wherein said micro ingredient solution comprises 35 to 150 grams per liter of Na₂ nitrilotriacetates, 25 to 100 grams per liter of MgCl₂-6H₂O, 6 to 30 grams per liter of FeSO₄-7H2O, 0.07 to 0.30 grams per liter of CoCl₂-6H₂O, 0.07 to 0.30 grams per liter of Na₂MoO₄-2H₂O, 0.15 to 0.60 grams per liter of NiCl₂-6H₂O, 0.01 to 0.1 grams per liter of Na₂SeO₃, 0.40 to 1.7 grams per liter Na₂WO₄-2H₂O

43. The system of claim 36, wherein said micro ingredient solution is prepared using deaerated water and maintained in anoxic condition in order to maintain iron ions as iron+3.

44. The system of claim 36, further comprising:
Said macro ingredient solution being prepared under normal atmospheric conditions and then deaerated;
Said micro ingredient solution being prepared under anoxic condition; and
Said micro ingredient solution being added to said deaerated macro ingredient solution.

45. The system of claim 44, wherein said micro ingredient solution is added to said deaerated macro ingredient solution at a ratio between 1 part micro ingredient solution to 100 to 400 parts macro ingredient solution.

46. The system of claim 44, wherein said micro ingredient solution is added to said deaerated macro ingredient solution at a ratio of 1 part micro ingredient solution to 250 parts macro ingredient solution.

47. The system of claim 34, wherein said media growth solution is directed into said reactor vessel through a media input port.

48. The system of claim 34. Wherein said media growth solution is maintained under a nitrogen blanket.

49. The system of claim 1, wherein said input material stream comprises at least in part carbon dioxide, a percentage of carbon dioxide being converted into biomass during exposure to said culture of methanogenic archea being less than 20 percent.

50. The system of claim 49, wherein excess biomass is selectively removed from said reactor vessel.

51. The system of claim 11, wherein said input material stream comprises at least in part carbon dioxide, a percentage of carbon dioxide being converted into biomass during exposure to said culture of methanogenic archea being less than 20 percent.

52. The system of claim 11, wherein said input material stream comprises at least in part carbon dioxide, a percentage of carbon dioxide being converted into biomass during exposure to said culture of methanogenic archea being between approximately 5 and 15 percent inclusive.

53. The system of claim 52, wherein excess biomass is selectively removed from said reactor through a biomass elimination port positioned on a lower portion of said reactor vessel.

54. The system of claim 5, wherein said pH adjustment system further comprises a pH buffer agent.

55. The system of claim 54, wherein said pH buffer agent comprises sodium hydroxide prepared using deaerated water and maintained under nitrogen until introduction into said reactor vessel.

56. The system of claim 54, wherein said pH buffer agent comprises potassium hydroxide prepared using deaerated water and maintained under nitrogen until introduction into said reactor vessel.

57. The system of claim 54, wherein said pH buffer agent comprises calcium hydroxide prepared using deaerated water and maintained under nitrogen until introduction into said reactor vessel.

58. The system of claim 54, wherein said pH buffer agent is prepared as approximately a 1.0 Normal solution.

59. The system of claim 54, wherein said pH buffer agent is prepared as less than a 1.0 Normal solution.

60. The system of claim 54, wherein said pH buffer agent comprises sodium bicarbonate.

61. The system of claim 54, wherein said pH buffer agent comprises ammonia.

62. The system of claim 54, wherein said pH buffer agent comprises ammonium.

63. The system of claim 54, wherein said pH buffer agent comprises ammonium nitrate.

64. The system of claim 11, wherein said pH adjustment system further comprises ammonium nitrate as a pH buffer agent.

65. The system of claim 1, wherein said input material stream is routed into said reactor vessel at a rate of 0.5 to 4.0 scfm per 5 cubic feet of reactor vessel volume.

66. The system of claim 1, wherein said input material stream is routed into said reactor vessel at a rate of 1.0 to 2.6 scfm per 5 cubic feet of reactor vessel volume.

67. The system of claim l,wherein said input material stream is routed into said reactor vessel and through at least one sparger positioned within said reactor vessel.

68. The system of claim 67, wherein each one of said at least one sparger creates bubbles approximately 1 to 10 microns in diameter.

69. The system of claim 1, wherein said input material stream is routed into said reactor vessel at a rate of approximately 1.9 to 2.6 scfm per 5 cubic feet of reactor vessel volume.

70. The system of claim 11, wherein said input material stream is routed into said reactor vessel at a rate of 0.5 to 4.0 scfm per 5 cubic feet of reactor vessel volume.

71. The system of claim 11, wherein said input material stream is routed into said reactor vessel at a rate of 1.0 to 2.6 scfm per 5 cubic feet of reactor vessel volume.

72. The system of claim 11, wherein said input material stream is routed into said reactor vessel at a rate of approximately 1.9 to 2.6 scfm per 5 cubic feet of reactor vessel volume.

73. The system of claim 11, wherein said input material stream is routed into said reactor vessel and through a sparger positioned within said reactor vessel.

74. The system of claim 73, wherein said sparger creates bubbles approximately 1 to 10 microns in diameter.

75. The system of claim 1, wherein said output material stream is generated at a rate of between 10 and 150 VVD.

76. The system of claim 1, wherein said output material stream is generated at a rate of between 35 and 100 VVD.

77. The system of claim 1, wherein said output material stream is generated at a rate of between 45 and 70 VVD.

78. The system of claim 11, wherein said output material stream is generated at a rate of between 10 and 150 VVD.

79. The system of claim 11, wherein said output material stream is generated at a rate of between 35 and 100 VVD.

80. The system of claim 11, wherein said output material stream is generated at a rate of between 45 and 70 VVD.

81. The system of claim 1, further comprising:
said input material stream comprising approximately four parts hydrogen to one part carbon dioxide;
wherein approximately 5 to 15% of the carbon dioxide is converted to cellular biomass through contact with the culture of methanogenic archea; and
wherein said output material stream comprises approximately 60 to 85% CH4.

82. The system of claim 81, wherein said output material stream further comprises hydrogen.

83. The system of claim 1, further comprising:
said input material stream comprising approximately 2 parts hydrogen to one part carbon dioxide;
wherein approximately 5 to 15% of the carbon dioxide is converted to cellular biomass through contact with the culture of methanogenic archea; and
wherein said output material stream comprises approximately 50 to 85% CH4.

84. The system of claim 83, wherein said output material stream further comprises carbon dioxide.

85. The system of claim 1, further comprising:
said input material stream comprising between approximately 2 parts hydrogen and approximately 5 parts hydrogen to one part carbon dioxide;
wherein approximately 5 to 15% of the carbon dioxide is converted to cellular biomass through contact with the culture of methanogenic archea; and
wherein said output material stream comprises approximately 50 to 85% CH4.

86. The system of claim 85, wherein said output material stream further comprises carbon dioxide.

87. The system of claim 85, wherein said output material stream further comprises hydrogen.

88. The system of claim 11, further comprising:
said input material stream comprising approximately four parts hydrogen to one part carbon dioxide;
wherein approximately 5 to 15% of the carbon dioxide is converted to cellular biomass through contact with the culture of methanogenic archea; and
wherein said output material stream comprises approximately 60 to 85% CH4.

89. The system of claim 88, wherein said output material stream further comprises hydrogen.

90. The system of claim 11, further comprising:
said input material stream comprising approximately 2 parts hydrogen to one part carbon dioxide;
wherein approximately 5 to 15% of the carbon dioxide is converted to cellular biomass through contact with the culture of methanogenic archea; and
wherein said output material stream comprises approximately 50 to 85% CH4.

91. The system of claim 90, wherein said output material stream further comprises carbon dioxide.

92. The system of claim 1, wherein said input material stream is directed through a gas filtering means prior to being directed into said reactor vessel.

93. The system of claim 92, wherein said gas filtering means further comprises a Water Gas Shift Reactor.

94. The system of claim 92, wherein said gas filtering means further comprise a Pressure Swing Adsorption Reactor.

95. The system of claim 92, wherein said gas filtering means further comprises a Vacuum Swing Adsorption Reactor.

96. The system of claim 92, wherein said gas filtering means further comprises a Membrane Filter.

97. The system of claim 96, wherein said membrane filter has a pore size between 2 and 10 Angstroms.

98. The system of claim 11, wherein said input material stream is directed through a gas filtering means prior to being directed into said reactor vessel.

99. The system of claim 98, wherein said gas filtering means further comprises a Water Gas Shift Reactor.

100. The system of claim 98, wherein said gas filtering means further comprise a Pressure Swing Adsorption Reactor.

101. The system of claim 98, wherein said gas filtering means further comprises a Vacuum Swing Adsorption Reactor.

102. The system of claim 98, wherein said gas filtering means further comprises a Membrane Filter.

103. The system of claim 100, wherein said membrane filter has a pore size between 2 and 10 Angstroms.

104. The system of claim 1, wherein said output material stream is directed into an output stream filtering means.

105. The system of claim 104, wherein said output stream filtering means further comprises a methane output and a recycling output, said recycling output being directed back into said reactor vessel.

106. The system of claim 104, wherein said output stream filtering means further comprises a water gas shift reactor.

107. The system of claim 104, wherein said output stream filtering means further comprises a Pressure Swing Adsorption Reactor.

108. The system of claim 104, wherein said output stream filtering means further comprises a Vacuum Swing Adsorption Reactor.

109. The system of claim 105, wherein said output stream filtering means further comprises a Membrane Filter.

110. The system of claim 109, wherein said Membrane Filter has a pore size between 2 and 10 Angstroms.

111. The system of claim 11, wherein said output material stream is directed into an output stream filtering means.

112. The system of claim 111, wherein said output stream filtering means further comprises a methane output and a recycling output, said recycling output being directed back into said reactor vessel.

113. The system of claim 111, wherein said output stream filtering means further comprises a water gas shift reactor.

114. The system of claim 111, wherein said output stream filtering means further comprises a Pressure Swing Adsorption Reactor.

115. The system of claim 111, wherein said output stream filtering means further comprises a Vacuum Swing Adsorption Reactor.

116. The system of claim 111, wherein said output stream filtering means further comprises a Membrane Filter.

117. The system of claim 109, wherein said Membrane Filter has a pore size between 2 and 10 Angstroms.

118. The system of claim 1, further comprising a thermal conditioning assembly operationally coupled to said reactor vessel.

119. The system of claim 118, wherein said thermal conditioning assembly maintains an internal temperature for said reactor vessel between 55 and 70 degrees Celsius.

120. The system of claim 118, wherein said thermal conditioning assembly maintains an internal temperature for said reactor vessel between 60 and 65 degrees Celsius.

121. The system of claim 11, further comprising a thermal conditioning assembly operationally coupled to said reactor vessel.

122. The system of claim 118, wherein said thermal conditioning assembly maintains an internal temperature for said reactor vessel between 55 and 70 degrees Celsius.

123. The system of claim 118, wherein said thermal conditioning assembly maintains an internal temperature for said reactor vessel between 60 and 65 degrees Celsius.

124. The system of claim 1, further comprising:
a second culture of methanogenic archea for converting and input material into an output material; and
a second reactor vessel operationally coupled between said input material stream and said output material stream in parallel with said reactor vessel, said second reactor vessel housing at least a portion of said second culture of methanogenic archea.

125. The system of claim 1, further comprising:
a plurality of second cultures of methanogenic archea for converting and input material into an output material; and
a plurality of second reactor vessels each operationally coupled between said input material stream and said output material stream in parallel with said reactor vessel, each one of said second reactor vessels housing at least a portion of an associated one of said plurality of second cultures of methanogenic archea.

126. The system of claim 11, further comprising:
a second culture of methanogenic archea for converting and input material into an output material; and
a second reactor vessel operationally coupled between said input material stream and said output material stream in parallel with said reactor vessel, said second reactor vessel housing at least a portion of said second culture of methanogenic archea.

127. The system of claim 11, further comprising:
a plurality of second cultures of methanogenic archea for converting and input material into an output material; and
a plurality of second reactor vessels each operationally coupled between said input material stream and said output material stream in parallel with said reactor vessel, each one of said second reactor vessels housing at least a portion of an associated one of said plurality of second cultures of methanogenic archea.

128. The system of claim 1, wherein said input material stream is directed into said reactor vessel and said output material stream is released from said reactor vessel in a continuous manner.

129. The system of claim 1, wherein said input material stream is directed into said reactor vessel periodically.

130. The system of claim 1, wherein said output material stream is released form said reactor vessel periodically.

131. The system of claim 1, wherein said input material stream is directed into said reactor vessel and said output material stream is released from said reactor vessel in a continuous manner.

132. The system of claim 1, wherein said input material stream is directed into said reactor vessel periodically.

133. The system of claim 1, wherein said output material stream is released form said reactor vessel periodically.

134. The system of claim 3, wherein said agitation system further comprises:
an agitation drive means; and
an impeller operationally coupled to said agitation drive means, said impeller positioned within said reactor vessel.

135. The system of claim 134, wherein said impeller rotates at between 1100 and 2100 rpm.

136. The system of claim 134, wherein said impeller rotates at between 1500 and 1800 rpm.

137. The system of claim 134, wherein said impeller rotates at greater than 110% of the resonance of the reactor vessel.

138. The system of claim 11, wherein said agitation system further comprises:
an agitation drive means; and
an impeller operationally coupled to said agitation drive means, said impeller positioned within said reactor vessel.

139. The system of claim 138, wherein said impeller rotates at between 1100 and 2100 rpm.

140. The system of claim 138, wherein said impeller rotates at between 1500 and 1800 rpm.

141. The system of claim 138, wherein said impeller rotates at greater than 110% of the resonance of the reactor vessel.

142. The system of claim 4, further comprising:
a growth media solution positioned within said reactor vessel and in contact with said culture of methanogenic archea;
said recirculation system selectively removing a portion of a combination of said culture of methanogenic archea and said growth media through a recirculation outlet port of said reactor vessel; and
said recirculation system returning said portion of said combination into said reactor through a recirculation inlet port of said reactor vessel.

143. The system of claim 142, wherein said selective removal and returning of said portion of said combination being done at a rate of between 5 and 50% of the reactor volume per hour.

144. The system of claim 142, wherein said selective removal and returning of said portion of said combination being done at a rate of between 10 and 20% of the reactor volume per hour.

145. The system of claim 1, wherein said culture of methanogenic archea comprises methanobacterim thermoautotrophicum or methanothermobacter thermautotrophicus.

146. The system of claim 1, wherein said culture of methanogenic archea comprises a thermophile.

147. The system of claim 1, wherein said culture of methanogenic archea comprises a xenophile.

148. The system of claim 11, further comprising:
wherein said output material stream is directed into an output stream filtering means;
wherein said output stream filtering means further comprises a methane output and a recycling output;
said recycling output being directed back into said reactor vessel;
said methane output being directed as an input into a specialty chemical processing facility.

149. A method of using methanogenic archea for the creation of useful products comprising the following steps:
providing a culture of methanogenic archea for converting an input material into an output material;
providing at least one input material stream;
providing at least one reactor vessel for housing at least a portion of the culture of methanogenic archea, each one of said at least one reactor vessel further comprises at least one input material stream port for operationally coupling said reactor vessel to a source of said input material stream and at least one output material stream port for facilitating removal of said output material stream;
providing at least one output material stream created at least in part by the culture of methanogenic archea;
providing an agitation system, said agitation system being at least partially positioned within said reactor vessel, said agitation system enhancing contact between said input material stream and said culture of methanogenic archea;
providing a recirculation system, said recirculation system enhancing contact between said input material stream and said culture of methanogenic archea;
providing a pH adjustment system, said pH adjustment system facilitating the maintenance of a pH of the methanogenic archea combined with a mixture of said input material stream and said output material stream;
providing a condenser environmentally coupled to said output material stream port, said condenser allowing a gaseous portion of said output material stream to be separated from a non-gaseous portion of said output material stream;
providing an input material stream flow control whereby the flow of the input material stream into said reactor vessel may be controlled;
providing a growth media solution;
providing a storage system;
placing a quantity of said growth media solution in said reactor vessel under substantially anerobic conditions;
placing a quantity of said methanogenic culture in said reactor vessel with said quantity of said growth media solution;
allowing said quantity of said methanogenic culture to grow for a predetermined period of time;
directing said input material stream into said reactor vessel;
bringing said input material stream into contact with said methanogenic archea for a predetermined period of time;
agitating a combination of said methanogenic archea, said input media stream and said growth media solution using said agitation system;
recirculating a portion of said combination of said methanogenic archea, said input media, and said growth media solution trough said recirculation system;
releasing a portion of said output material stream through said output material stream port into said condenser;
directing an output of said condenser into said storage system.

150. The method of claim 149, wherein said step of providing a growth media solution further comprises the following steps:
providing a macro ingredient solution, said macro ingredient solution comprising KH₂PO₄, NH₄CL, and NaCl prepared under normal atmospheric conditions;
deaerating said macro ingredient solution;
providing a micro ingredient solution, said micro ingredient solution comprising Na₂ nitrilotriacetates, MgCl₂-6H₂O, FeSO₄-7H₂O, CoCl₂-6H₂O, Na₂MoO₄-2H₂O, NiCl₂-6H₂O, Na₂SeO₃, Na₂WO₄-2H₂O prepared under anoxic condition; and
adding said micro ingredient solution to said deaerated macro ingredient solution at a ratio between 1 part micro ingredient solution to 100 to 400 parts macro ingredient solution.

151. The method of claim 149, wherein said step of providing a pH adjustment system further comprises the step of providing at least one pH buffer agent selected from the group of buffer agents consisting of sodium hydroxide, potassium hydroxide, calcium hydroxide, sodium bicarbonate, ammonia, ammonium, and ammonium nitrate.

152. The method of claim 149, wherein said step of directing said input material stream into said reactor vessel further comprises routing said input material stream into said reactor vessel at a rate of 0.5 to 4.0 scfm per 5 cubic feet of reactor vessel volume.

153. The method of claim 149, wherein said step of releasing a portion of said output material stream further comprises releasing said output material stream at a rate of between 35 and 100 VVD.

154. The method of claim 149, wherein said step of providing at least one reactor vessel further comprises the following steps: providing a plurality of reactor vessels; and
configuring said plurality of reactor vessels in parallel between said input material stream and said output material stream.

155. The method of claim 149, wherein said step of providing a culture of methanogenic archea further comprises providing a plurality of methanogenic cultures.
